(19) United States
(12) Patent Application Publication
(10) Pub. No.: US 2008/0038367 A1
(43) Pub. Date: Feb. 14, 2008

(54) Nutritional supplement compositions and methods of preparing

(76) Inventor: Sal Saloum, Morristown, NJ (US)

Correspondence Address: MCCARTER & ENGLISH LLP;CITYPLACE I, 185 ASYLUM STREET, HARTFORD, CT, 06103, US

(21) Appl. No.: 11/503,475

(22) Filed: Aug. 11, 2006

(51) Int. Cl. A61K 36/752; A61K 36/45; A61K 36/53; A61K 36/534; A61K 36/328; A61K 36/48; A61K 36/28

(52) U.S. Cl. 424/617; 424/732; 424/764; 424/736; 424/745; 424/747; 424/757; 424/769; 424/748; 424/638; 424/646; 424/702; 424/724; 424/778

(57) ABSTRACT

The present invention relates to a nutritional supplement composition and methods for preparing. The invention further provides a method for creating a nutrient supplement drink composition comprising the a carrier liquid, a flower distillate, and at least one herbal extract component, and optionally including an additional ingredient selected from the group consisting of a second herbal extract component, a nutrient, a flavoring agent, a preservative, a coloring agent, a second carrier agent, and combinations thereof.

### FIELD OF THE INVENTION

The present invention relates to nutritional supplement compositions containing antioxidants, which are useful for improving health and preventing disease when consumed by an individual. More particularly, the present invention relates to nutritional supplement drink compositions comprising a combination of herbal extracts.

### BACKGROUND OF THE INVENTION

Oxidative damage to DNA, lipids, proteins, and other macromolecules is considered to be one of the most important causes of inflammatory and degenerative diseases. Specifically, studies have shown that reactive oxygen species (ROS) or oxygen free radicals, for example, singlet oxygen, superoxide, peroxyl radicals, hydroxyl radicals and peroxynitrite, generated by many biochemical pathways are a fundamental component of cellular and tissue oxidative damage in many human diseases. In fact, free radicals have been implicated in over 50 diseases, and it has been estimated that at least 85% of chronic and degenerative diseases result from oxidative damage caused by free radicals.

Antioxidants are compounds that protect cells against the damaging effects of reactive oxygen species. An imbalance between antioxidants and reactive oxygen species results in oxidative stress, leading to cellular damage and many chronic health problems, such as cancer; atherosclerosis; myocardial infarction; arthritis; immune diseases, inflammation, lupus, and scleroderma; neurodegenerative diseases such as Alzheimer's Disease and Parkinson's Disease; acquired immune deficiency syndrome (AIDS); cataracts; ischemic injury; skin wrinkling; and generalized aging. For example, the oxidation of low-density lipoprotein (LDL) has been recognized to play an important role in atherosclerosis. LDL oxidation can be induced by macrophages and can also be catalyzed by metal ions like copper. Macrophages recognize and engulf oxidized LDL, a process that leads to the formation of atherosclerotic plaques in the arterial wall.

The human body has several mechanisms to counteract damage by free radicals and other reactive oxygen species. One important mechanism that guards against free radical damage is the action of antioxidants, which act as radical scavengers and convert the radicals to less reactive species. Many types of antioxidants are not generated endogenously and must be ingested through consumption of nutrient containing foods, like fruits and vegetables.

Plants have long been used in traditional medicines for the treatment and prevention of diseases. Even today, it is estimated that approximately two-thirds of the world's population rely on medicinal plants as the primary source of medicines. In recent years, the physiological effects of foods (e.g., fruits, vegetables, nuts, and chocolate) and beverages (e.g., fruit juices, wine, tea, and coffee) rich in polyphenolic compounds have generated great interest in the scientific community. As dietary sources of biologically active compounds, these foods prove to be valuable for health. The physiological benefit imparted by the polyphenolic compounds centers around their ability to reduce reactive oxygen species or oxygen free radicals. Over the past several decades many studies have focused on the antioxidant activity exhibited by these plant-derived phenolic compounds and their capacity to treat or prevent cardiovascular disorders, diabetes, blood pressure, inflammatory diseases, and high cholesterol.

In particular, recent studies have demonstrated that flavonoids found in various herbs, flowers, fruits, and vegetables act as powerful antioxidants. Flavonoids, or bioflavonoids, are a ubiquitous group of polyphenolic substances which are present in most plants, concentrating in seeds, fruit skin or peel, bark, and flowers. The beneficial effects of fruit, vegetables, and tea or even red wine have been attributed to flavonoid compounds rather than to known nutrients and vitamins. Flavonoids help provide protection against oxidative stress and disease by contributing, along with antioxidant vitamins and enzymes, to the total antioxidant defense system of the human body. In fact, the potent antioxidant activity of flavonoids and their ability to scavenge hydroxyl radicals, superoxide anions, and lipid peroxy radicals may be their most important function, and underlies many of the above actions in the body. Epidemiological studies have shown that flavonoid intake is inversely related to mortality from coronary heart disease and to the incidence of heart attacks, and that certain flavonoids can protect LDL from being oxidized and prevent atherosclerosis. In addition, a great number of plant medicines contain flavonoids, which have been reported by many authors as having antibacterial, anti-inflammatory, antiallergic, antimutagenic, antiviral, antineoplastic, anti-thrombotic, and vasodilatory actions.

The capacity of flavonoids to act as antioxidants depends upon their molecular structure. The structural components common to these molecules include two benzene rings on either side of a 3-carbon ring. Multiple combinations of hydroxyl groups, sugars, oxygens, and methyl groups attached to these structures create the various classes of flavonoids: flavanols, flavanones, flavones, flavan-3-ols (catechins), anthocyanins, and isoflavones. The position of hydroxyl groups and other features in the chemical structure of flavonoids are important for their antioxidant and free radical scavenging activities. Quercetin, the most abundant dietary flavonol, is a potent antioxidant because it has all the right structural features for free radical scavenging activity.

Due to the fact that oxidative damage is implicated in many disease processes, and laboratory research on flavonoids and other antioxidants suggests that they are useful in the prevention and treatment of a number of these disorders, there exists a general need to improve the quality of food ingested in the human body and to fortify various antioxidative mechanisms in the body. In view of the foregoing, it will be appreciated that providing a nutritional supplement drink comprising herbal extracts rich in antioxidants and other nutrients will provide greater health benefits to consumers.

### SUMMARY OF THE INVENTION

The present invention provides compositions and methods for producing a nutritional supplement comprising a combination of plant-derived extracts rich in nutrients, for example, antioxidants, volatile or essential oils, minerals, and vitamins. Because the herbal-extract components contain different nutrient components at different concentrations, they are expected to work synergistically to improve the health of an individual when administered in the particular combinations of the present invention because they have different, but complementary, mechanisms of action.

In one aspect the present invention relates to a nutritional supplement that comprises extracts from a combination of antioxidant-rich herbs, plants, and flowers. In certain embodiments, the invention comprises a nutritional supplement drink composition comprising a rose flower extract, for example, rose flower distillate, and/or orange blossom distillate together with at least one other herbal extract selected from the group consisting of chamomile flower, orange blossom, tilia flower, rose mallow flower, chrysanthemum flower, black caraway, marigold, wolf berry, green cardamom, mint, sumac, clove, sage, bergamot, lemon, lime, hyssop, jasmine, and balm. As used herein, “distillate” may refer to the process or the product of distillation.

When consumed by an individual, the herbal supplement of the invention may be useful for improving general health, or treating and/or preventing diseases and conditions such as cancer; atherosclerosis; myocardial infarction; arthritis; immune diseases; inflammation; lupus; and scleroderma; neurodegenerative diseases such as Alzheimer's Disease and Parkinson's Disease; acquired immune deficiency syndrome (AIDS); cataracts; ischemic injury; skin wrinkling; gastrointestinal disorders; viral, bacterial or microbial infections, sun or UV exposure, and conditions associated with generalized aging.

In any of the embodiments described herein, the nutritional supplement of the invention comprises an antioxidant, for example, a bioflavonoid, and optionally includes a nutrient component; for example, a mineral, a vitamin, a lipid, an oil, a protein, an amino acid, or a nucleic acid; a flavoring agent; a preservative; a coloring agent; a carrier; or a combination thereof.

In one aspect the present invention relates to methods for preparing an nutritional supplement composition, comprising the steps of; (1) providing a volume of a carrier liquid; (2) providing a flower distillate component; (3) providing an herbal extract component, for example, an antioxidant containing herbal extract; and (4) admixing the herbal extract, and the flower distillate with the liquid carrier, and wherein the nutritional supplement composition optionally comprises an additional ingredient selected from the group consisting of a nutrient, for example, a mineral, a vitamin, a lipid, an oil; a protein, an amino acid, a nucleic acid or combination thereof; a flavoring agent; a preservative; a coloring agent; a second carrier agent; and combinations thereof. In any of the preferred embodiments, the carrier agent comprises water, oil, alcohol or a combination thereof. As will be understood by one of ordinary skill, the precise combinations and amounts of flower distillate and herbal extracts can be varied in any number of ways according to any of the methods of the invention described herein, to achieve the desired flavor and/or nutritional composition, and the examples and embodiments described herein are given by way of nonlimiting example only.

In another embodiment of the methods of the invention, the method for preparing a nutritional supplement composition, comprises the steps of; (1) providing a volume of a carrier liquid; (2) providing a flower distillate component; (3) providing a suitable amount of an herb, for example, an antioxidant containing herb; (4) placing the herbal component in the carrier liquid and heating to a temperature of from about 40° C. to about 200° C. for a duration of from about 10 seconds to about one hour; (5) allowing the herbal component to steep in the carrier liquid for a duration of from about 1 minute to about 48 hours, and wherein heat is no longer actively applied to the carrier liquid; and (6) filtering the herbal component pulp from the carrier liquid, and wherein the flower distillate is added to the carrier liquid either prior to boiling or after filtering. In certain embodiments, the nutritional supplement further comprises the addition of an additional ingredient selected from the group consisting of a nutrient, for example, a mineral, a vitamin, a lipid, an oil, a protein, an amino acid, a nucleic acid; a flavoring agent; a preservative; a coloring agent; a second carrier agent; and combinations thereof. In any of the preferred embodiments, the carrier agent comprises water, oil, alcohol or a combination thereof. As will be understood by one of ordinary skill, the precise combinations and amounts of flower distillate and herbal extracts can be varied in any number of ways according to any of the methods of the invention described herein, to achieve the desired flavor and/or nutritional composition, and the examples and embodiments described herein are given by way of nonlimiting example only.

Preferably, the herbal extract is prepared in a solvent or carrier liquid comprising distilled water, oil, alcohol or a combination thereof. In certain embodiments the alcohol is ethanol. In other embodiments, the nutritional supplement comprises a semi-solid through the addition of a thickening agent, for example, a glycol, a sugar, a cellulose, or binder. In certain preferred embodiments, the nutritional supplement is prepared using water, for example, distilled water, and demonstrates viscosity similar to that of water, such that it is convenient for oral consumption by an individual as a drink. In still other embodiments, the nutritional supplement of the invention has a viscosity of from about 0.3×10⁻³ Pa·s to about 1.0 Pa·s. In any of embodiments disclosed herein, the nutritional supplement drink can additionally be consumed as a hot or cold beverage.

The nutritional supplement compositions of the present invention comprise a flower distillate, for example, rose flower water, orange blossom water, or combinations thereof, together with an effective amount of an herbal extract. In certain embodiments, the herbal extracts of the present invention are prepared separately in smaller volumes of solvent or carrier and admixed together to result in the final combination. In other aspects of the invention, the herbal ingredients are combined in a suitable volume of solvent or carrier and the nutritional supplement is created in a one-step extraction process.

Before the present nutritional supplement and methods are disclosed and described, it is to be understood that this invention is not limited to the particular configurations, process steps, and materials disclosed herein as such configurations, process steps, and materials may vary somewhat. The following detailed examples are given by way of example of the preferred embodiments, and are in no way considered to be limiting to the invention. For example, the relative quantities of the ingredients may be varied to optimize the desired effects, additional ingredients may be added, and/or similar ingredients may be substituted for one or more of the ingredients described. Additional advantageous features and functionalities associated with the systems, methods and processes of the present invention will be apparent from the detailed description which follows.

### DETAILED DESCRIPTION OF THE INVENTION

As used herein, the term “extract” is used generally and encompasses herbal derived aqueous extracts, non-aqueous extracts, distillates, and infusions unless expressly and specifically indicated otherwise, which are biologically and therapeutically active.

As used herein, “nutrient” refers to a biologically and therapeutically active substance that, taken into a human or animal, serves to help sustain it in its existence, promoting growth and repair, replacing loss, and/or providing energy. As it is used in the present disclosure, nutrient, encompasses without limitation, antioxidants, vitamins, minerals, lipids, oils, proteins, amino acids, lipids, carbohydrates, nucleic acids, and the like.

As used herein, “herb” or “herbal” is used generally to refer to a plant and/or part of a plant including the seed, root, stem, leaf, flower, and/or fruit that contains compounds of medicinal, nutritional, gustatory, and/or aromatic value.

The term “herb extract” is used generally and in reference to an extract, as defined herein, obtained from a whole plant or any part of the plant indicated, including, for example, the seed, root, stem, leaf, flower, fruit (if any) and/or combinations thereof. For example, when referring to an extract of black caraway, the invention contemplates use of the seed, root, stem, leaf, flower, fruit, and combinations thereof in preparation of the extract. Furthermore, in any of the preferred embodiments described herein, it is expressly contemplated that the compositions and methods of the invention encompass the use of fresh and/or dried herbs.

As used herein, the phrases “herbal drink,” “nutritional supplement,” and “nutritional supplement drink” are used interchangeably, and refer to the compositions of the invention that contain plant-extract derived antioxidants and nutrients that work synergistically to improve the health of an individual when orally consumed.

As used herein, the term “drink” refers generally to any liquid or semi-solid form suitable for oral consumption by an individual that is now known or becomes known by those skilled in the art, and also includes concentrates, for example, frozen concentrates, and freeze-dried powders that can be dissolved in a suitable volume of liquid carrier to generate a, so called, “instant” liquid or semi-solid for consumption by an individual. In certain embodiments the nutritional drink of the invention have a viscosity of from about 0.3×10⁻³ Pa·s to about 1.0 Pa·s.

In one aspect the present invention relates to methods for preparing an nutritional supplement composition, comprising the steps of; (1) providing a volume of a carrier liquid; (2) providing a flower distillate component; (3) providing an herbal extract component, for example, an antioxidant or other nutrient containing herb; and (4) admixing the herbal extract, and the flower distillate with the liquid carrier, and wherein the nutritional supplement composition optionally comprises an additional ingredient selected from the group consisting of a nutrient, for example, a mineral, a vitamin, a lipid, an oil; a protein, an amino acid, a nucleic acid or combination thereof; a flavoring agent; a preservative; a coloring agent; a second carrier agent; and combinations thereof. In any of the preferred embodiments, the carrier agent comprises water, oil, alcohol or a combination thereof. In certain embodiments, the flower distillate comprises from about 0.05% to about 60% by weight (i.e., w/v) of the nutritional supplement volume. In other embodiments, the herbal extract comprises from about 0.05% to about 99% (w/v) of the nutritional supplement. In a preferred embodiment, the flower distillate comprises from about 0.1% to about 20% (w/v), and the herbal extract comprises from about 0.5% to about 60% (w/v) of the nutritional supplement. As will be understood by one of ordinary skill, the precise combinations and amounts of flower distillate and herbal extracts can be varied in any number of ways according to any of the methods of the invention described herein, to achieve the desired flavor and/or nutritional composition, and the examples and embodiments described herein are given by way of non-limiting example only.

In another embodiment of the methods of the invention, the method for preparing a nutritional supplement composition, comprises the steps of; (1) providing a volume of a carrier liquid; (2) providing a flower distillate component; (3) providing a suitable amount of an herb, for example, an antioxidant or other nutrient containing herb; (4) placing the herbal component in the carrier liquid and heating to a temperature of from about 40° C. to about 200° C. for a duration of from about 10 seconds to about one hour; (5) allowing the herbal component to steep in the carrier liquid for a duration of from about 1 minute to about 48 hours, and wherein heat is no longer actively applied to the carrier liquid; and (6) filtering the herbal component pulp from the carrier liquid, and wherein the flower distillate is added to the carrier liquid either prior to boiling or after filtering. In certain embodiments, the nutritional supplement further comprises the addition of an additional ingredient selected from the group consisting of a nutrient, for example, a vitamin, a lipid, an oil, a protein, an amino acid, a nucleic acid; a flavoring agent; a preservative; a coloring agent; a second carrier agent; and combinations thereof. In any of the preferred embodiments, the carrier agent comprises water, oil, alcohol or a combination thereof. In certain embodiments, the flower distillate comprises from about 0.05% to about 60% by weight (i.e., w/v) of the nutritional supplement volume. In other embodiments, the total amount of antioxidant containing herb comprises from about 0.5 grams to about 60 grams per liter. In a preferred embodiment, the flower distillate comprises from about 0.1% to about 20% (w/v) of the nutritional supplement, and the amount of herb comprises from about 1 gram to about 25 grams per liter. In another preferred embodiment, the amount of herb comprises from about 5 grams to about 20 grams per liter. As will be understood by one of ordinary skill, the precise combinations and amounts of flower distillate and herbal extracts can be varied in any number of ways according to any of the methods of the invention described herein, to achieve the desired flavor and/or nutritional composition, and the examples and embodiments described herein are given by way of non-limiting example only.

In any of the preferred embodiments, the flower distillate comprises rose flower distillate, orange blossom distillate or a combination thereof.

In any of the embodiments described herein, the herb is selected from the group consisting of chamomile, orange blossom, marigold, wolf berry, tilia, rose mallow, chrysanthemum, black caraway, cardamom, mint, sumac, clove, sage, bergamot, lemon, lime, hyssop, jasmine, balm, and combinations thereof. Certain embodiments of the methods or compositions described herein include a mix of herbs independently selected from the list above. It should be recognized that the particular amount of each type of herb used to create the nutritional supplement can be varied in any number of combinations depending on the desired taste and nutritional composition, all of which are expressly contemplated as being within the scope of the present invention.

In other embodiments described herein, the herbal extract comprises an extract of an herb selected from the group consisting of chamomile, orange blossom, marigold, wolf berry, tilia, rose mallow, chrysanthemum, black caraway, cardamom, mint, sumac, clove, sage, bergamot, lemon, lime, hyssop, jasmine, balm, and combinations thereof. In certain embodiments of the methods or compositions described herein include a mix of herbs independently selected from the list above. It should be recognized that the particular amount of each type of herbal extract used to create the nutritional supplement can be varied in any number of combinations depending on the desired taste and nutritional composition, all of which are expressly contemplated as being within the scope of the present invention.

In any of the preferred embodiments, the herbal extract is prepared in a carrier, for example, oil, water, ethanol, or combinations thereof. Most preferably, the herbal extract is prepared using distilled water.

The herbal-based nutritional supplement drink compositions of the present invention are composed of flower distillate, for example, rose flower water distillate, and/or orange blossom water distillate, and the synergistic mixture of the extracts. As described above, the herbal extracts of the present invention can be created extracted separately or together using the above processes.

In another embodiment, the invention includes a method for preparing an herbal extract, comprising the steps of; (1) providing a volume of a carrier liquid; (2) providing a suitable amount of an herb, for example, an antioxidant or other nutrient containing herb; (3) placing the herbal component in the carrier liquid and heating to a temperature of from about 40° C. to about 200° C. for a duration of from about 10 seconds to about one hour; (4) allowing the herbal component to steep in the carrier liquid for a duration of from about 1 minute to about 48 hours, and wherein heat is no longer actively applied to the carrier liquid; and (6) filtering the herbal component pulp from the carrier liquid, and wherein the flower distillate is added to the carrier liquid either prior to boiling or after filtering. In a preferred embodiment the amount of herb used to create the extract is from about 0.5 grams to about 60 grams per liter.

In any of the preferred embodiments, after the extraction step the herb or plant pulp is filtered from the solvent or carrier by passing the extract through one or more filters. In certain embodiments, the method of the invention comprises a two-step filtration process: a first filtration step is performed by passing the crude extract (i.e., extract plus herb pulp material) through a first filter, or crude extract filter, to remove larger sized material. One of ordinary skill in the art will recognize that any suitable filter can be used as long as the herb pulp material is retained in the filter and the extract solvent or carrier is able to pass through the filter into a clean receptacle. By way of non-limiting example, suitable filter materials for the crude filter include, a colander, cheesecloth, paper towel, and the like. The resulting primary filtrate is then subjected to a second filter step preformed by passing the volume of primary filtrate recovered from the first filter step through a second filter to remove the finer particulates. The purpose of the second filter step is to remove substantially all pulp material from the extract filtrate. There are many choices for use in the second filter step that are known to those of skill in the art and include, for example, coffee filters, vacuum filters, microfilters such as those available from Millipore™, PUR™, Brita™, and Katadyn™. In certain other embodiments, the herbal extract or extracts are filtered in a one-step process. In a preferred embodiment, the single filter step substantially removes all herb pulp material from the final filtrate. In a preferred embodiment, the extract is passed through a filter with a fine mesh suitable for removal of contaminants, for example, bacteria so that the nutritional supplement is essentially sterile, which will be safer for consumption by an individual, and will also have a longer shelf-life.

In other aspects the invention relates to a nutritional supplement created according to the method of the invention which is concentrated in a reduced volume suitable for convenient and/or long term storage, for example, by freezing or freeze-drying, for later consumption such as by dissolution in suitable volume of a carrier liquid, for example, water, alcohol, juice, and the like. In certain embodiments, the concentrate supplement can be used as a topping, additive, sauce, or marinate for other foods and/or drinks to improve the taste and nutritional value thereof.

In other aspects, the nutritional supplement drink can be used to create an alcoholic beverage. In certain embodiments, the supplement is used as a “mixer” with a suitable volume of an alcohol-containing carrier. In still other embodiments, the supplement drink is fermented in the presence of yeast and optionally includes additional sugar to create an alcoholic beverage. The fermentation may be followed by a subsequent distillation step to produce a distilled spirit.

In any of the embodiments, the nutritional supplement may additionally comprise dissolved carbon dioxide, i.e., carbonation, to create a soda beverage. Soda's are popular and are pleasant tasting, refreshing, and have certain medicinal values. In any of the preferred embodiments, the nutritional supplement drink may be carbonated artificially, for example, by dissolving carbon dioxide gas in the liquid at high pressure, or may be naturally carbonated, for example, as result of yeast fermentation of sugar.

In still another embodiment, the nutrient supplement of the invention is used as a flavoring for other foods, for example, ice cream, and other frozen treats. The methods for making ice cream is well known in the art.

In still another embodiment, the nutritional supplement may be concentrated and administered in a pill, capsule, lozenge, etc. . . . and taken in one or more doses per day. Methods of encapsulating nutritional supplements as liquids, gels, powders, and the like are widely known by those of skill in the art.

When consumed by an individual, the herbal drink of the invention may be useful for improving general health, or treating and/or preventing diseases and conditions such as cancer; atherosclerosis; myocardial infarction; arthritis; immune diseases; inflammation; lupus; and scleroderma; neurodegenerative diseases such as Alzheimer's Disease and Parkinson's Disease; acquired immune deficiency syndrome (AIDS); cataracts; ischemic injury; skin wrinkling; and conditions associated with generalized aging.

In any of the embodiments the herbal nutritional supplement drink of the invention may additionally comprise at least one of the following nutrients: a mineral, for example, calcium, magnesium, chromium, copper, iodine, iron, manganese molybdenum, selenium, zinc, boron, sodium, potassium, silicon, and mixtures thereof; one or more nutrients, for example, a carotinoids, beta-carotene, lutein, lycopene, choline, para-aminobenzoic acid, alpha-lipoic acid, coenzyme Q10, a phospholipid, linoleic acid, linolenic acid, inositol, methylsulfonyl methane, spirulina, and mixtures thereof; a vitamin, for example, vitamin A, vitamin C, vitamin D, vitamin E, vitamin K, vitamin B-12, thiamine, riboflavin, niacin, pantothenic acid, pyridoxine, folic acid, biotin, derivatives thereof, and mixtures thereof; a lipid; one or more amino acids, for example, arginine, histidine, lysine, isoleucine, leucine, methionine, phenylalanine, threonine, tryptophan, tyrosine, valine, aspartate, glutamate, serine, proline, asparagine, glutamine, cysteine, glycine, alanine, and mixtures thereof; a flavoring agent, for example, anethole, anise oil, benzaldehyde, blackberry, blueberry, caraway, caraway oil, cardamom oil, cardamom seed, cherry juice, cherry syrup, cinnamon, cinnamon oil, cinnamon water, citric acid, citric acid syrup, clove oil, cocoa, coriander oil, dextrose, eriodictyon, ethyl acetate, ethyl vanillin, fennel oil, ginger, glucose, glycerin, glycyrrhiza, grape, honey, lavender oil, lemon oil, lime, mannitol, methyl salicylate, myristica oil, orange oil, orange peel, orange syrup, peppermint, peppermint oil, peppermint water, phenylethyl alcohol, pineapple, raspberry juice, raspberry syrup, rosemary oil, rose oil, rose water, sarsaparilla syrup, sorbitol, spearmint, spearmint oil, strawberry, sucrose, fructose, fruit juice, thyme oil, tolu balsam, vanilla, vanillin, and wild cherry syrup. Additional information on flavoring agents may be obtained in Food Chemicals Codex (5^{th} ed. 2003) and Fenaroli's Handbook of Flavor Ingredients (5^{th} ed. 2004); and combinations thereof.

In other embodiments, the invention relates to a nutritional supplement drink created according to any of the methods of the invention described herein.

Following is a description of flowers and herbs of the invention. A more detailed description of the herbs can be found in, Bown. D., Encyclopaedia of Herbs and their Uses. Dorling Kindersley, London. 1995 (ISBN 0-7513-020-31), which is incorporated herein by reference in its entirety for all purposes.

Rose flower water (Rosa Damascena). The rose water is steam distilled from fresh Bulgarian Roses (Rosa Damascena) and contains essential oils and antioxidants. Rose water maintains the pH balance, stimulates regeneration processes, has a calming effect; treats inflammation.

Chamomile. Chamomile's active ingredients are volatile oils, tannic acids, and glucosides. The primary active ingredient of the essential oil from Chamomile is bisabolol. Chamomile's health benefits include antispasmodic, stomach ache, cramps, and indigestion, IBS, colic, mucositis, heartburn, improves appetite, gout, headache, diuretic and tonic or sleep aid, emmanagogue, sedative, sooths fevers, inflammation, neuralgia, antiseptic. The extract or infusion is made by adding desired amount of the flowers to boiling water and allowing to steep for about 30 minutes in a covered vessel, in order to prevent the escape of steam, as the medicinal value of the flowers is to a considerable extent impaired by any evaporation, and the infusion should be allowed to stand on the flowers for about 10 minutes before straining off.

Tilia is a genus of about 30 species of trees, native throughout most of the temperate Northern Hemisphere, in Asia (where the greatest species diversity is found), Europe and eastern North America; it is absent from western North America. The tree produces fragrant and nectar-producing flowers, the medicinal herb lime blossom. The flower extract is useful as a nervine, and is used to treat restlessness, hysteria, and headaches.

Rose mallow. The active constituents of rose mallow extract include asparagine, althein, ascorbic-acid, flavonol glycosides (including gossypin-3-sulfate), malvin, pectin, phenolic-acids, quercetin (anti-inflammatory, anti-oxidant, anti-cancer agent), salicylic-acid, and sucose. Mallows are analgesic, antitussive, demulcent, diuretic, febrifuge, highly emollient, slightly laxative and odontalgic. Mallow or Hibiscus tea is known in alternative medicine for its use as a demulcent to soothe throat inflammations and laryngitis, as an expectorant for coughs and bronchitis. It is used in the treatment of dysentery, lung ailments and urinary ailments. The tea is also taken for gastritis and enteritis or used as an enema for intestinal inflammations, and is used as an antipyretic and to reduce blood pressure.

Marigold. The Marigold is a native of south Europe but hardy, and easy to grow. Seeds sown in April, in any soil, in sunny, or half-sunny places germinate freely. They require no other cultivation but to keep them clean from weeds and to thin out where too close, leaving them 9 to 10 inches apart, so that their branches may have room to spread. The plants will begin to flower in June, and continue flowering until the frost kills them. The seeds ripen in August and September, and if permitted to scatter will furnish a supply of young plants in the spring. Either the whole flowerheads or just the ray-florets are used medicinally. Among the constituents are an essential oil, pigments (carotenoids, for example, lutein and zeaxanthins), bitter compounds, saponins, flavonoid glycosides, mucilage and resin. These give Marigold vulnerary, anti-inflammatory, choleretic and antispasmodic properties. Lutein and Zeaxanthins have also been shown to useful for the prevention and treatment of macular degeration.

Wolf Berry. Wolf berry contains polysaccharides(amylose), detaine, beta-sitosterol, lutein, zeaxanthine, physalein, ascorbic acid, carotene, riboflavin, nicotine, thiamine, betaine, taurine, vitamin E, vitamin C, vitamin B1, vitamin B2, 19 kinds of amino acids and K, Na, Ca, Mg, Fe, Cu, Mn, Zn, Se, 21 kinds of minerals, protein, and fat. The fruit is taken internally in the treatment of high blood pressure, diabetes, poor eyesight, vertigo, lumbago, impotence and menopausal complaints. The fruit is harvested when fully ripe and is dried for later use. The root bark is a bitter, cooling, antibacterial herb that controls coughs and lowers fevers, blood pressure and blood cholesterol levels. It is taken internally in the treatment of chronic fevers, internal haemorrhages, nosebleeds, tuberculosis, coughs, asthma etc. It is applied externally to treat genital itching. The bark is harvested in the winter and dried for later use. The plant has a long history of medicinal use, both as a general, energy restoring tonic and also to cure a wide range of ailments from skin rashes and eyesight problems to diabetes. The fruit of many members of this genus is a very rich source of vitamins and minerals, especially in vitamins A, C and E, flavanoids and other bio-active compounds. It is also a fairly good source of essential fatty acids, which is fairly unusual for a fruit. It is being investigated as a food that is capable of reducing the incidence of cancer and also as a means of halting or reversing the growth of cancers.

Chrysanthemum. Chrysanthemum has many purported medicinal uses, including an aid in recovery from influenza, and as an anti-inflammatory. According to traditional Chinese medicine it can aid in the prevention of sore throat and promote the reduction of fever. Derived from the flower and aerial parts of the plant, chrysanthemum increases coronary vasodilatation and coronary blood flow, but has little effect on cardiac contractility or oxygen consumption. Chrysanthemum reduces capillary permeability induced by histamine and operates as an antibacterial and antipyretic agent. It is also used to treat hypertension, angina, dizziness, dry eye, headache, and cough. Its active ingredients include essential oils like chrysantheonon, camphor, and bornol; alkaloids: stachydrine; sesquiterpenes: alantolactone; glycosides: Acacetin-7-rhamnoglucose, cosmosin, acacetin-7-glucose, diosmetin-7-glucose; and other compounds: adenine, and choline.

Black Caraway. The oil is rich in nutrients including essential fatty acids, vitamins, minerals, various monosaccharides, 8 of the 9 essential amino acids, carotenoids, non-starch polysaccharides, sterol/sterolins, etc. There are actually more than a hundred substances many of which are unique. It is not known whether individual substances, e.g. nigellin, are responsible for its healing effects or, as with most herbs, the combination does the job; it is an immunomodulatory; anti-inflammatory; bronchodilator; increases bile flow in the liver and helps to expel stones. The seeds contain numerous esters of structurally unusual unsaturated fatty acids with terpene alcohols (7%); furthermore, traces of alkaloids are found which belong to two different types: isochinoline alkaloids are represented by nigellimin and nigellimin-N-oxide, and pyrazol alkaloids include nigellidin and nigellicin. In the essential oil thymoquinone was identified as the main component (up to 50%) besides p-cymene (40%), α-pinene (up to 15%), dithymoquinone and thymohydroquinone. Other terpene derivatives were found only in trace amounts: carvacrol, carvone, limonene, 4-terpineol, citronellol. Furthermore, the essential oil contains significant (10%) amounts of fatty acid ethyl esters. The seeds also contain a fatty oil rich in unsaturated fatty acids, mainly linoleic acid (50-60%), oleic acid (20%), eicodadienoic acid (3%) and dihomolinoleic acid (10%) which is characteristic for the genus. Saturated fatty acids (palmitic, stearic acid) amount to about 30% or less. Commercial nigella oil (“Black Seed Oil”, “Black Cumin Oil”) may also contain parts of the essential oil, mostly thymoquinone, by which it acquires an aromatic flavor. Black Cumin (Nigella sativa) Seed is rich in nutritional values. Monosaccharides (single molecule sugars) in the form of glucose, rhamnose, xylose, and arabinose are found in the black seed. The Black Cumin (Nigella sativa) Seed contains a non-starch polysaccharide component which is a useful source of dietary fiber. It also contains compounds such as ascorbic acid, quercetin and luteolin in the tea. Ascorbic acid has antioxidant qualities, which means it eliminates free radicals, and research indicates it also supports the immune system, along with helping prevent strokes, cancer and heart attacks. Rutin, another compound found in rooibos, reinforces and stabilizes blood vessels. Several carboxylic acids isolated in the tea have the potential to inhibit damage to the liver.

Cardamom. Cardamom is used to treat infections in teeth and gums, to prevent and treat throat troubles, congestion of the lungs and pulmonary tuberculosis, inflammation, and also digestive disorders. It is also reportedly used as an antidote for both snake and scorpion venom; to treat stomach-aches, constipation, dysentery, and other digestion problems. Cardamom has a strong, unique taste, with an intensely aromatic fragrance. One of the most expensive spices by weight, little is needed to impart the flavor. Cardamom is best stored in pod form, because once the seeds are exposed or ground, they quickly lose their flavor. The content of essential oil in the seeds is strongly dependent on storage conditions, but may be as high as 8%. In the oil were found α-terpineol 45%, myrcene 27%, limonene 8%, menthone 6%, β-phellandrene 3%, 1,8-cineol 2%, sabinene 2% and heptane 2%. (Phytochemistry, 26, 207, 1987); Other sources report 1,8-cineol (20 to 50%), α-terpenylacetate (30%), sabinene, limonene (2 to 14%) and borneol.

Sumac. The sumac bush, Rhus coriara, has sour, astringent, red-colored berries that are regarded as a spice flavor, being similar to tamarind. The plant is a member of the cashew family; it grows in Mediterranean. Medical uses have included digestion and bowel problems. It is said to have diuretic and antipyretic properties. Its medicinal qualities are wholly to be ascribed to its stypticity or astringency.

Clove. The clove tree (Syzgium aromaticum, or Eugenia caryophyllis) is also indigenous to the Moluccas, and the clove of commerce is the dried unexpanded flower. The principle constituent of cloves, and the one to which their properties are essentially due, is the oil. Unlike most spices, clove has an obvious medical value. It contains eugenol which is an effective local anesthetic, and this has long been used in dentistry. Other constituents include salicylic acid. Cloves in substance or infusion are sometimes given to relieve nausea and vomiting, more especially the vomiting of pregnancy, to relieve flatulence, and to except weak digestion.

Hyssop. Hyssop is an evergreen, bushy herb, growing 1 to 2 feet high, with square stem, linear leaves and flowers in whorls, six- to fifteen-flowered. Hyssop is cultivated for the use of its flower-tops, which are steeped in water to make an infusion, which is sometimes employed as an expectorant, diaphoretic, stimulant, pectoral, and carminative. The healing virtues of the plant are due to a particular volatile oil, which is stimulative, carminative and sudorific. It admirably promotes expectoration, soothes stomach ache, asthma, muscular rheumatism, and is anti-setpic. It is also considered a powerful vermifuge, relaxes peripheral blood vessels, promotes sweating, anti-inflammatory, anti-catarrhal, and antispasmodic. Its active constituents are volatile oil, flavonoids, tannins and marrubin. The essential oils from hyssop is a gently relaxing nerve tonic suitable for treating nervous exhaustion, anxiety and depression. Hyssop leaves can be preserved by drying. They should be harvested on a dry day at the peak of their maturity and the concentration of active ingredients is highest. They should be dried quickly, away from bright sunlight in order to preserve their aromatic ingredients and prevent oxidation of other chemicals. Good air circulation is required, such as an airing cupboard with the door left open, or a sunny room, aiming for a temperature of 20-32° C. Hyssop leaves should dry out in about six days, any longer and they will begin to discolour and lose their flavour. The dried leaves are stored in clean, dry, labelled airtight containers, and will keep for 12-18 months.

Jasmine. Jasmine (Jasminum) is a genus of shrubs and vines in the Family Oleaceae, with about 200 species, native to tropical and warm temperate regions. Jasmine is a climbing, vine-like plant better known for its aromatic flowers and essential oil. Perhaps not so well known however, is Jasmine's traditional use for calming the nerves, and for lending its delightful fragrance & flavor to teas. Jasmine's medicinal uses include for coughs and respiratory ailments.

Bergamot. Bergamot is an aromatic herb native to eastern North America. The whole plant is strongly impregnated with a delightful fragrance; even after the darkly-coloured leaves have died away, the surface rootlets give off the pleasant smell by which the plant has earned its common name ‘Bergamot,’ it being reminiscent of the aroma of the Bergamot Orange.

Spearmint. Spearmint is the common garden mint is originally a native of the Mediterranean region. The chief constituent of Spearmint oil is carvone. There are also present phellandrine, limonene and dihydrocarveol acetate. Esters of acetic, butyric and caproic or caprylic acids are also present. (An Ester is a combination of an alcohol with an acid, the combination being associated with the elimination of water. The esters are highly important and in many cases dominant constituents of numerous essential oils.). The properties of Spearmint oil resemble those of Peppermint, being stimulant, carminative and antispasmodic, but its effects are less powerful, and it is less used than Peppermint, though it is better adapted for children's maladies. A Spearmint extract will relieve hiccough, indigestion, fever, and inflammatory diseases.

Peppermint. Among essential oils, peppermint ranks first in importance. The chief constituent of peppermint extract is menthol, but it also contains menthyl acetate and isovalerate, together with menthone, cineol, inactive pinene, and limonene. The principal ester constituent, menthyl acetate, possesses a very fragrant minty odor, to which the agreeable aroma of the extract is largely due. The alcoholic constituent, Menthol, possesses the well-known penetrating minty odor and characteristic cooling taste. The flavoring properties of the extract are due largely to both the ester and alcoholic constituents, while the medicinal value is attributed to the latter only. Peppermint oil is the most extensively used of all the volatile oils, both medicinally and commercially. The characteristic anti-spasmodic action of the volatile oil is more marked in this than in any other oil, and greatly adds to its power of relieving pains arising in the alimentary canal. It may also be employed for other sudden pains and for cramp in the abdomen; wide use is made of Peppermint in cholera and diarrhea. Peppermint allays sickness and nausea, and is much used to disguise the taste of unpalatable drugs, as it imparts its aromatic characteristics to whatever prescription it enters into.

Menthol is used in medicine to relieve the pain of rheumatism, neuralgia, throat affections and toothache. It acts also as a local anesthetic, vascular stimulant and disinfectant. It is inhaled for chest complaints, and nasal catarrh, laryngitis or bronchitis are often alleviated by it. It is also used internally as a stimulant or carminative. On account of its anesthetic effect on the nerve endings of the stomach, it is of use to prevent sea-sickness. The fresh leaves of the plant will, relieve local pains and headache, act as an antipyretic, and an aperient.

Sage. The chief constituent of Sage and its active principle is a yellow or greenish-yellow volatile oil. Tannin and resin are also present in the leaves, 0.5 to 1.0 percent of the oil is yielded from the leaves and twigs when fresh, and about three times this quantity when dry. Sage oil contains a hydrocarbon called Salvene; pinene and cineol are probably present in small amount, together with borneol, a small quantity of esters, and the ketone thujone, the active principle which confers the power of resisting putrefaction in animal substances. Dextro-camphor is also present in traces. It also contains salviol, which is now known to be excellent for soothing the throat and tonsils, and also for ulcerated throat.

Sage extract is a valuable agent in the relief from fever, treating disorders of the stomach, and nervous system and weakness of digestion generally. It was for this reason that the Chinese valued it, giving it the preference to their own tea. It is considered a useful medicine in typhoid fever and beneficial in biliousness and liver complaints, kidney troubles, haemorrhage from the lungs or stomach, for colds in the head as well as sore throat and quinsy and measles, for pains in the joints, lethargy and palsy. Sage extract contains essential oils such as pinene, cineol, and linalool.

Balm. Balm (or balsam) is native to South Europe, especially in mountainous regions. Balm extract is a carminative, diaphoretic and febrifuge. It induces a mild perspiration and makes a pleasant and cooling tea for feverish patients in cases of catarrh and influenza. Balm is a useful herb, either alone or in combination with others. It is used to treat colds and fever.

Lemon. Lemon contains essential oils, such as limonene, and antioxidants such as hesperitin. Lemon juice is probably the best of all antiscorbutics, being almost a specific in scurvy, and it is valuable as a cooling drink in fevers, and for allaying thirst. The juice may be used in diaphoretic and diuretic draughts. It is highly recommended in acute rheumatism, and is sometimes given to counteract narcotic poisons, especially opium. Locally, it is a good astringent, whether as a gargle in sore throat, hiccough, and is helpful in jaundice and palpitation of the heart.

Lime Fruit. The Lime is a small tree, crooked and prickly, only reaching a height of 8 feet. The flowers are small and white and the fruit about half the size of a lemon, with a smoother, thinner rind, having a greenish tinge in its yellow. The juice is principally used in the manufacture of citric acid, and for medicinal purposes is often used indiscriminately with that of the lemon, although its flavor is not so popular. The rind contains a volatile oil including the terpene limonene and citral. Lime is used as an antiscorbutic.

Honey is a sweet and viscous fluid produced by honeybees and other insects from the nectar of flowers. Liquid honey does not spoil. Because of its high sugar concentration, it kills bacteria by plasmolysis. Natural airborne yeasts cannot become active in it because the moisture content is too low. Natural, raw honey varies from 14% to 18% moisture content. As long as the moisture content remains under 18%, virtually no organism can successfully multiply to significant amounts in honey. For around 2000 years, honey has been used to treat a variety of ailments, though it was not until modern times that the cause of infection was understood. Now, modern research into the use of honey as an antimicrobial agent has revealed potential treating a variety of ailments. Antibacterial properties of honey are the result of the low water activity causing osmosis, hydrogen peroxide effect, and high acidity. Honey is primarily a saturated mixture of two monosaccharides. This mixture has a low water activity; most of the water molecules are associated with the sugars and few remain available for microorganisms, so it is a poor environment for their growth. Honey is a mixture of sugars and other compounds. The specific composition of any batch of honey will depend largely on the mix of flowers consumed by the bees that produced the honey. Honey has a density of about 1500 kg/m³ (50% denser than water) which means 12-13 pounds per gallon. Typical honey analysis Fructose: 38% Glucose: 31% Sucrose: 1% Water: 17% Other sugars: 9% (maltose, melezitose) Ash: 0.17%.

Flavonoids. Flavonoids are widely distributed in plants fulfilling many functions including producing yellow or red/blue pigmentation in flowers and protection from attack by microbes and insects. The widespread distribution of flavonoids, their variety and their relatively low toxicity compared to other active plant compounds (for instance alkaloids) mean that many animals, including humans, ingest significant quantities in their diet. Flavonoids have been found in high concentrations in butterflies and moths sequestered from dietary intake at the larval stage and then stored in adult tissues. Flavonoids have been referred to as “nature's biological response modifiers” because of strong experimental evidence of their ability to modify the body's reaction to allergens, viruses, and carcinogens. They show anti-allergic, anti-inflammatory, anti-microbial and anti-cancer activity. In addition, flavonoids act as powerful antioxidants, protecting against oxidative and free radical damage.

Over 5000 naturally occurring flavonoids have been characterized from various plants. They have been classified according to their chemical structure, and are usually subdivided into 6 subgroups: flavonols, such as, quercetin, kaempferol, myricetin, isorhamnetin; flavones, such as, luteolin, and apigenin; flavanones, such as, hesperitin, naringenin, and eriodictyol; flavan-3-ols, such as, catechin, gallocatechin, epicatechin, epigallocatechin, epicatechin 3-gallate, epigallocatechin 3-gallate, theaflavin, theaflavin 3-gallate, theaflavin 3′-gallate, theaflavin 3,3′ digallate, and thearubigins; isoflavones, such as, genistein, daidzein, and glycitein; and anthocyanidins, such as, cyanidin, delphinidin, malvidin, pelargonidin, peonidin, petunidin.

It is believed that flavonoids act by inhibiting hormones, such as estrogen, that may trigger hormone-dependent malignancies like cancers of the breast, endometrium, ovary, and prostate. Studies show that quercetin, a flavonoid found in citrus fruits, can block the spread of cancer cells in the stomach. Flavonoids also stabilize mast cells, a type of immune cell that releases inflammatory compounds, like histamine, when facing foreign microorganisms. Histamine and other inflammatory substances are involved in allergic reactions. Mast cells are large cells present in connective tissue. Flavonoids fortify and repair connective tissue by promoting the synthesis of collagen. Collagen is a remarkably strong protein of the connective tissue that “glues” the cells together. Flavonoids are believed to benefit connective tissue and reduce inflammation.

Catechin and its derivatives, oligomeric proanthocyanidins, quercetin and quercetin chalcone, Ginkgo flavone glycosides, silymarin, and others can be utilized in preventative and treatment protocols for cardiovascular disease, cancer, inflammatory conditions, asthma, periodontal disease, liver disease, cataracts and macular degeneration.

Quercetin is a flavonoid that forms the “backbone” for many other flavonoids, including the citrus flavonoids rutin, hesperidin, naringin and tangeritin. In studies, quercetin is found to be the most active of the flavonoids, and many medicinal plants owe much of their activity to their high quercetin content. Quercetin has demonstrated significant anti-inflammatory activity because of direct inhibition of several initial processes of inflammation. For example, it inhibits both the manufacture and release of histamine and other allergic/inflammatory mediators. In addition, it exerts potent antioxidant activity and vitamin C-sparing action.

Quercetin chalcone (QC), a novel flavonoid, is quercetin with an opened C ring and the oxygen found in the C-ring of quercetin converted into a hydroxyl group. QC also retains the C-ring double bond from quercetin, and should retain the antioxidant properties of quercetin as well. In fact, with the addition of the extra hydroxyl group in the C ring, quercetin chalcone could be a more potent antioxidant than quercetin. Quercetin chalcone is also more water soluble than quercetin (unpublished data), which should increase QC's absorption and bioavailability over quercetin.

Proanthocyanidins extracts demonstrate a wide range of pharmacological activity. Their effects include increasing intracellular vitamin C levels, decreasing capillary permeability and fragility, scavenging oxidants and free radicals, and inhibiting destruction of collagen, the most abundant protein in the body. Oligomeric proanthocyanidins (OPCs, pycnogenols) are oligomeric flavonoids, usually dimers and trimers, based on the flavan-3-ol, or catechin, molecule, sometimes attached to gallic acid. Commercial sources of OPCs include grape seeds and pine bark. However, bilberry, cranberry, black currant, green tea, black tea, and other plants also contain these flavonoids.

Supplementation with OPC has been shown to protect against cardiovascular and other degenerative diseases and to have numerous other benefits, including: lowering LDL cholesterol levels, reducing platelet aggregation, increasing the strength and elasticity of blood vessels, helping collagen repair itself, reducing edema and inflammation, relieving functional problems associated with varicose veins, lessening the tendency toward diabetic retinopathy, and improving skin health.

Cigarette smoke, air pollution, alcohol, drugs, radiation from televisions and computers, chemicals and a busy life generate free radicals, which cause “oxidative stress”. Free radicals attack all your vital cellular structures, such as cell membranes and stimulate processes that have been linked to accelerated cellular aging. An antioxidant's job is to neutralize the free radical cells thus protecting the cells in our body from accelerated aging. By neutralizing the free radicals, these once free radical cells are able to assist with nutritional distribution, proper circulation and overall better health.

Epicatechin improves blood flow and thus seems good for cardiac health. Even in very high amounts (for example, 140 grams per day), flavonoids do not appear to cause unwanted side effects. Even when raised to the level of 10% of total caloric intake, flavonoid supplementation has been shown non-toxic. Studies during pregnancy have also failed to show problems with high-level intake of flavonoids. Most flavonoids function in the human body as antioxidants. In this capacity, they help neutralize overly reactive oxygen-containing molecules and prevent these overly reactive molecules from damaging parts of cells. Particularly in oriental medicine, plant flavonoids have been used for centuries in conjunction with their antioxidant, protective properties. Scultellaria root, comus fruit, licorice, and green tea are examples of flavonoid-containing foods widely used in oriental medicine. While flavonoids may exert their cell structure protection through a variety of mechanisms, one of their potent effects may be through their ability to increase levels of glutathione, a powerful antioxidant, as suggested by various research studies.

As used herein, “vitamin D” includes all of its active forms including, for example, ergocalciferol, cholecalciferol, and mixtures thereof. Similarly, “vitamin E” includes all of its active forms including, for example, alpha-tocopherol, beta-tocopherol, gamma-tocopherol, delta-tocopherol, and mixtures thereof. Similarly, “vitamin A” includes all of its active forms including, for example retinol, dehydroretinol, retinoic acid, and mixtures thereof. Similarly, “vitamin K” includes all of its active forms including, for example, phylloquinone, vitamin farnoquinone, menadione or menaquinone, synthetic analogs of menadione, and mixtures thereof. Similarly, “vitamin B-12” includes all of its active forms including, for example, cyanocobalamin, methylcobalamin, hydroxocobalamin, nitritocobalamin, and mixtures thereof. As used herein, “derivatives” of vitamins means alternative, biologically active forms of a particular vitamin. For example, derivatives of vitamin E include esters of vitamin E, such as d-alpha-tocopheryl acetate. As another example, derivatives of vitamin A include esters of vitamin A, such as retinyl palmitate. As still another example, derivatives of niacin include niacinamide. As yet another example, derivatives of pyridoxine include pyridoxal and pyridoxamine. As a still further example, derivatives of vitamins that are acids include salts of such acids, for example, calcium ascorbate, thiamine hydrochloride, pyridoxine hydrochloride, calcium pantothenate, and the like.

Vitamins are organic compounds that are required for the normal growth and maintenance of life of animals, including humans, who are generally unable to synthesize these compounds by anabolic processes that are independent of environment other than air, and which compounds are effective in small amounts, do not furnish energy, and are not utilized as building units for the structure of the organism, but are essential for the transformation of energy and for the regulation of the metabolism of structural units. Vitamins or their precursors are found in plants, and thus plant tissues are the sources for the animal kingdom of these protective nutritional factors. In addition to carbohydrates, fats, proteins, mineral salts, and water, it is essential that the food of humans and animals contain small amounts of these vitamins. If any one of at least 13 of these compounds is lacking in the diet, a breakdown of the normal metabolic processes occurs, which results in a reduced rate or complete lack of growth in children and in symptoms of malnutrition that are classified as deficiency diseases.

The functions of vitamins generally fall into two categories, the maintenance of normal structure and the maintenance of normal metabolic functions. For example, vitamin A is essential for the maintenance of normal epithelial tissue, and vitamin D functions in the absorption of normal bone salts for the formation and growth of a sound bony structure. Certain vitamins, such as thiamine, riboflavin, pantothenic acid, and niacin, are known to be essential constituents of the respiratory enzymes that are required in the utilization of energy from oxidative catabolism of sugars and fats.

It is convenient to divide vitamins into two groups, the water-soluble vitamins and the fat-soluble vitamins. The water-soluble vitamins include ascorbic acid and the B group of vitamins, which comprises some 10 or more well-defined compounds. The fat-soluble vitamins include vitamins A, D, E, and K, since they can be extracted with organic solvents and are found in the fat fractions of animal tissues. For brief reviews of vitamins in general and specific vitamins, see Remington's Pharmaceutical Sciences.

Choline has the property of preventing the deposition of excess fat, or of causing the removal of excess fat from the liver of experimental animals fed high-fat diets and, because of this, is often classified as a “lipotropic agent.” The lipotropic action probably relates to the incorporation of choline into phosphatidyl choline (lecithin), which, in turn, is incorporated into phospholipids and lipoproteins, which are critical for normal membrane structure and function. The lipotropic action is independent of the function of choline as a reservoir of methyl groups.

In the body choline is mainly found in phospholipids, such as lecithin (phosphatidylcholine) and sphingomyelin. The outer leaflet of plasma membrane is rich in these choline-phospholipids whereas the inner leaflet is dominated by phosphatidylethanolamine, phosphatidylserine, and phosphatidylinositol. Phosphatidylcholine, the predominant phospholipid (>50%) in most mammalian membranes, not only contributes to the structure of the membrane bilayer, but products of receptor-mediated lecithin hydrolysis also serve as important second messengers in signal cascades that control cell growth and gene expression. Disaturated phosphatidylcholine is the primary active component of surfactant in the lung; a deficiency of surfactant in the neonate leads to respiratory distress syndrome in premature infants.

The metabolism of choline, methionine, and methyl-folate are closely interrelated; the metabolic pathways intersect at the formation of methionine from homocysteine. Some choline can be formed from methionine (through the methylation of phosphatidylethanolamine by phosphatidylethanolamine N-methyltransferase using S-adenosylmethionine as the methyl donor). This can provide some of the choline required by humans.

Para-aminobenzoic acid (PABA) is part of the coenzyme tetrahydrofolic acid. As such, it aids in the utilization of amino acids, supports red blood cell formation, and assists in the manufacture of folic acid in the intestines. It has been linked to hair growth, as well as reversing the graying of hair, but these results are disappointing. People suffering from vitiligo, over-pigmentation of skin, or without pigment in some spots, have reported an improvement of the skin after more PABA was ingested.

Minerals serve a wide variety of essential physiological functions ranging from structural components of body tissues to essential components of many enzymes and other biological important molecules. Minerals are classified as micronutrients or trace elements on the basis of the amount present in the body. The seven micronutrients (calcium, potassium, sodium, magnesium, phosphorus, sulphur, and chloride) are present in the body in quantities of more than five grams. Trace elements, which include boron, copper, iron, manganese, selenium, and zinc are found in the body in quantities of less than five grams.

Micronutrient Minerals. Calcium is the mineral element believed to be most deficient in the diet in the United States. Calcium intakes in excess of 300 mg per day are difficult to achieve in the absence of milk and dairy products in the diet. This is far below the recommended dietary allowance (RDA) for calcium (1000 mg per day for adults and children ages one to ten, 1200 mg per day for adolescents and pregnant and lactating women, which equates to about four glasses of milk per day). In fact, it has been reported that the mean daily calcium intake for females over age 12 does not exceed 85 percent of the RDA. In addition, during the years of peak bone mass development (18 to 30), more than 66 percent of all U.S. women fail to consume the recommended amounts of calcium on any given day. After age 35, this percentage increases to over 75 percent. Calcium is needed for formation and maintenance of bones, the development of teeth, and maintaining healthy gums. Calcium is also necessary for blood clotting and stabilizing many body functions, and is thought to assist in preventing bowel cancer.

Magnesium is the second most plentiful cation of the intracellular fluids. It is essential for the activity of many enzyme systems and plays an important role with regard to neurochemical transmission and muscular excitability. Deficits are accompanied by a variety of structural and functional disturbances. The average 70-kg adult has about 2000 mEq of magnesium in the body. About 50% of this magnesium is found in bone, 45% exists as an intracellular cation, and 5% is in the extracellular fluid. About 30% of the magnesium in the skeleton represents an exchangeable pool present either within the hydration shell or on the crystal surface. Mobilization of the cation from this pool in bone is fairly rapid in children, but not in adults. The larger fraction of magnesium in bone is apparently an integral part of bone crystal. Magnesium is a cofactor of all enzymes involved in phosphate transfer reactions that utilize adenosine triphosphate (ATP) and other nucleotide triphosphates as substrates. Various phosphatases and pyrophosphatases also represent enzymes from an enormous list that are influenced by this metallic ion.

Magnesium plays a vital role in the reversible association of intracellular particles and in the binding of macromolecules to subcellular organelles. For example, the binding of messenger RNA (mRNA) to ribosomes is magnesium dependent, as is the functional integrity of ribosomal subunits. Certain of the effects of magnesium on the nervous system are similar to those of calcium. An increased concentration of magnesium in the extracellular fluid causes depression of the central nervous system (CNS). Hypomagnesemia causes increased CNS irritability, disorientation, and convulsions. Magnesium also has a direct depressant effect on skeletal muscle. Abnormally low concentrations of magnesium in the extracellular fluid result in increased acetylcholine release and increased muscle excitability that can produce tetany. Magnesium helps with formation of teeth and bones and assists the absorption of calcium and potassium. Where calcium stimulates the muscles, magnesium relaxes the muscles. Magnesium is further needed for cellular metabolism and the production of energy through its help with enzyme activity. It is used for muscle tone of the heart and assists in controlling blood pressure. Together with vitamin B-12, magnesium may prevent calcium oxalate kidney stones, depression, dizziness, muscle twitching, and pre-menstrual syndrome (PMS). It can help prevent the calcification of soft tissue and may help prevent cardiovascular disease, osteoporosis, and certain forms of cancer, and it may reduce cholesterol levels.

Sodium is an electrolyte in the body and is required for the manufacture of hydrochloric acid in the stomach, which helps to protect the body from infections.

Potassium is needed for growth, building of muscles, transmission of nerve impulses, heart activity, and other body processes.

While not generally classified as either a micronutrient or a trace element, silicon is important for maintaining health of bones, cartilage, tendons, and artery walls. Silicon may also be beneficial in the treatment of allergies, heartburn, and gum disease, as well as promoting a healthy immune system. Silicon is also required for keeping nails, hair, and skin in good condition and is useful in counteracting the effects of aluminum.

Trace Elements. Boron is required by the body in trace amounts for proper metabolism of calcium, magnesium, and phosphorus, as well as vitamin D. Boron helps brain function, healthy bones, and can increase alertness. Boron is also useful for people who want to build muscle. Boron is known to help prevent postmenopausal osteoporosis. Further, a relationship has been shown between a lack of boron in the diet and the chances of developing arthritis. R. E. Newnham, 46 Journal of Applied Nutrition (1994).

Chromium is an important trace element wherein the lack of sufficient chromium in the diet leads to impairment of glucose utilization, however, disturbances in protein and lipid metabolism have also been observed. Impaired glucose utilization occurs in many middle-aged and elderly human beings. In experimental studies, significant numbers of such persons have shown improvement in their glucose utilization after treatment with chromium. Chromium is transported by transferrin in the plasma and competes with iron for binding sites. Chromium as a dietary supplement may produce benefits due to its enhancement of glucose utilization and its possible facilitating the binding of insulin to insulin receptors, which increases its effects on carbohydrate and lipid metabolism. It functions with glucose tolerance factor (GTF) when this hormone-affiliated enters the bloodstream because of an increase of insulin. Chromium as a supplement may produce benefits in atherosclerosis, diabetes, rheumatism, and weight control.

Copper is another important trace element in the diet. The most common defect observed in copper-deficient animals is anemia. Other abnormalities include growth depression, skeletal defects, demyelination and degeneration of the nervous system, ataxia, defects in pigmentation and structure of hair or wool, reproductive failure, and cardiovascular lesions, including dissecting aneurisms. Several copper-containing metalloproteins have been isolated, including tyrosinase, ascorbic acid oxidase, laccase, cytochrome oxidase, uricase, monoamine oxidase, .delta.-aminolevulinic acid hydrydase, and dopamine-.beta.-hydroxylase. Copper functions in the absorption and utilization of iron, electron transport, connective tissue metabolism, phospholipid formation, purine metabolism, and development of the nervous system. Ferroxidase I (ceruloplasmin), a copper-containing enzyme, effects the oxidation of Fe(II) to Fe(III), a required step for mobilization of stored iron. A copper-containing enzyme is thought to be responsible for the oxidative deamination of the epsilon amino group of lysine to produce desmosine and isodesmosine, the cross-links of elastin. In copper-deficient animals the arterial elastin is weaker and dissecting aneurisms may occur. Copper is required in the formation of hemoglobin, red blood cells, and bones, while it helps with the formation of elastin and collagen, thus making it necessary for wound healing. Copper is also a constituent of superoxide dismutase (SOD), a powerful enzyme that scavenges free radicals in cells.

Iodine is important for the production of thyroid hormones, which regulate cellular oxidation. The iodine-deficiency disease is goiter. In iodine-deficient young, growth is depressed and sexual development is delayed, the skin and hair are typically rough, and the hair becomes thin. Cretinism, feeble-mindedness, and deaf-mutism occur in a severe deficiency. There is reproductive failure in females and decreased fertility in males that lack sufficient iodine in the diet. Iodine-containing thyroid hormones regulate the conversion of fat to energy, thus stabilizing body weight and controlling cholesterol levels.

Iron is an essential component of several important metalloproteins. These include hemoglobin, myoglobin, and many oxidation-reduction enzymes. In iron deficiency, there may be reduced concentrations of some of the iron-containing enzymes, such as cytochrome c in liver, kidney, and skeletal muscle, and succinic dehydrogenase in the kidney and heart. Thus, iron is needed for oxygenation of red blood cells, a healthy immune system, and energy production. Iron is a component of several peroxidase enzymes and cofactors of these enzymes. Therefore, iron is involved in scavenging of hydrogen peroxide radicals.

Manganese plays a role in the synthesis of glycosaminoglycans (GAGs), collagen, and glycoproteins, which are important constituents of cartilage and bone. Manganese is required for enzyme activity of glycosyltransferases. This family of enzymes is responsible for linking sugars together into GAGs, adding sugars to other glycoproteins, adding sulfate to aminosugars, converting sugars to other modified sugars, and adding sugars to lipids. These functions are manifested as GAG synthesis (hyaluronic acid, chondroitin sulfate, karatan sulfate, heparin sulfate, and dermatin sulfate, among others), collagen synthesis, and function of many other glycoproteins and glycolipids. GAGs and collagen are chief structural elements for all connective tissues. Their synthesis is essential for proper maintenance and repair of connective tissues.

Manganese deficiencies in humans and animals lead to abnormal bone growth, swollen and enlarged joints, and slipped tendons. In humans, manganese deficiencies are associated with bone loss, arthritis, and impaired glucose utilization. Levels of all GAGs are decreased in connective tissues during manganese deficiencies, with chondroitin sulfates being most depleted. Manganese-deficient organisms quickly normalize GAG and collagen synthesis when manganese is provided.

Manganese is also required for activity of manganese superoxide dismutase (MnSOD), which is present only in mitochondria. Manganese deficiency decreases the activity of MnSOD and may lead to mitochondrial dysfunction, manifested as decreased cellular functions. MnSOD is a powerful enzyme that scavenges free radicals in mitochondria. It is also believed that MnSOD assists in preventing diabetes and is needed for normal nerve function. Manganese is required for the conversion of mevalonic acid to squalene. Pyruvate carboxylase is a manganese metalloenzyme, which is repressible by insulin and important in the citric acid cycle for the oxidation of carbohydrates, lipids, and proteins, as well as in the synthesis of glucose and lipids. Manganese also enables the body to utilize vitamin C, vitamin B-1, and biotin, as well as choline. Manganese is used in the manufacture of fat, sex hormones, and breast milk. Manganese also is thought to important in brain functioning.

Molybdenum is an essential mineral found in highest concentrations in the liver, kidneys, skin, and bones. This mineral is required by the body to properly metabolize nitrogen. It is also a vital component of the enzyme xanthine oxidase, which is required to convert purines to uric acid, a normal byproduct of metabolism. Molybdenum also supports the body's storage of iron and other cellular functions, such as growth. A deficiency of molybdenum is associated with mouth and gum disorders and cancer. A diet high in refined and processed foods can lead to a deficiency of molybdenum, resulting in anemia, loss of appetite and weight, and stunted growth in animals. While these deficiencies have not been observed directly in humans, it is known that a molybdenum deficiency can lead to impotence in older males. Molybdenum also assists in breaking down sulfite toxin build-up in the body, and may prevent cavities. These qualities suggest that molybdenum possesses antioxidant properties. Further, molybdenum assists the body in fighting nitrosamines, which are associated with certain cancers.

Selenium is an essential trace element that functions as a component of enzymes involved in protection against free radicals and in thyroid hormone metabolism. In several intra- and extra-cellular glutathione peroxidases and iodothyronine 5′-deiodinases, selenium is located at the active centers as the selenoamino acid, selenocysteine (SeCYS). At least two other proteins of unknown function also contain SeCYS. Although SeCYS is an important dietary form, it is not directly incorporated into these specific selenium-proteins; instead, a co-translational process yields tRNA-bound SeCYS. In contrast, selenium as seleno-methionine is incorporated non-specifically into many proteins, as it competes with methionine in general protein synthesis. Therefore, tissues often contain both specific, as well as the nonspecific, selenium-containing proteins when both SeCYS and selenomethionine are consumed, as found in many foods. Selenium is a major antioxidant nutrient and is involved in protecting cell membranes and preventing free radical generation, thereby decreasing the risk of cancer and disease of the heart and blood vessels. Medical surveys show that increased selenium intake decreases the risk of breast, colon, lung, and prostate cancer. Selenium also preserves tissue elasticity; slows down the aging and hardening of tissues through oxidation; and helps in the treatment and prevention of dandruff. Recent research has shown antitumorigenic effects of high levels of selenium in the diets of several animal models. Moreover, selenium helps rid the body of toxic heavy metals, such as mercury, lead, and cadmium. Selenium also stimulates increased antibody response to infections, promotes more energy in the body, alleviates menopausal symptoms in women, and promotes production of healthy sperm in males. In certain cases, selenium has proven effective in fighting cold sores and shingles, both of which are caused by herpes virus.

Zinc is known to occur in many important metalloenzymes. These include carbonic anhydrase, carboxypeptidases A and B, alcohol dehydrogenase, glutamic dehydrogenase, D-glyceraldehyde-3-phosphate dehydrogenase, lactic dehydrogenase, malic dehydrogenase, alkaline phosphatase, and aldolase. Impaired synthesis of nucleic acids and proteins has been observed in zinc deficiency. There is also evidence that zinc may be involved in the secretion of insulin and in the function of the hormone. Zinc is also necessary for a healthy immune system and is useful for treating skin conditions, such as acne and boils, and for treating sore throats. Zinc is also needed for cell division and for growth and maintenance of muscles. Children need zinc in the diet for normal growth and sexual development. Zinc is also a constituent of superoxide dismutase (ZnSOD), which scavenges free radicals. Further, zinc is required for growth and maintenance of hair, nails, and skin.

According to the present invention, minerals can be provided as inorganic compounds, such as chlorides, sulfates, iodides, and the like. In addition, some minerals can be provided in more bioavailable forms, such as amino acid chelates, which are well known in the art. U.S. Pat. No. 5,292,538. Examples of minerals that can be provided as amino acid chelates include calcium, magnesium, manganese, zinc, iron, boron, copper, molybdenum, chromium, and silicon. Still further, minerals can be provided as organic compounds, such as ascorbates, citrates, picolinates, aspartates, carbonates, bicarbonates, and the like. Illustrative examples of various mineral forms according to the present invention include potassium bicarbonate, sodium bicarbonate, calcium carbonate, calcium ascorbate, zinc picolinate, manganese picolinate, copper aspartate, molybdenum trioxide, chromium picolinate, potassium iodide, boron citrate, silicon amino acid chelate, and the like.

Carotenoids are a family of hundreds of plant pigments found in fruits and vegetables that are red, orange, and deep yellow in color, and also in some dark green leafy vegetables. See USDA-NCC Carotenoid Database for U.S. Foods (1998). Carotenoids are the precursors of most of the vitamin A found in animals. At least 10 different carotenoids exhibit provitamin A activity, including alpha.- and .beta.-carotenes and cryptoxanthin. As precursors of vitamin A, carotenoids exhibit an effect on vision, but carotenoids are known to have other beneficial effects in the diet, as well. For example, carotenoids are also known for their antioxidant activity in helping protect the body from free radical damage.

Volumes of research reveal that two carotenoids—lutein and zeaxanthin—are found in great concentrations in the macula of the eye. This research also indicates that maintaining high levels of these two carotenoids, especially lutein, may help diminish the effects of age-related macular degeneration, the leading cause of blindness in those over 65 years of age. Lutein acts as an antioxidant, protecting cells against the damaging effects of free radicals. As with the other carotenoids, lutein is not made in the body and, therefore, must be obtained from food or dietary supplements.

At one time researchers believed all antioxidants served the same purpose. Now there is growing evidence that individual antioxidants may be used by the body for specific purposes. Researchers believe that lutein is deposited into areas of the body most prone to free radical damage. One major example is the macula, a tiny portion of the retina. Research indicates that because of its antioxidant properties, lutein consumption may play a role in maintaining the health of the eyes, heart and skin as well as the breasts and cervix in women. In addition, scientists are studying lutein's possible role in age-related macular degeneration, cataracts, heart disease, and immune system health. Studies have also shown that lutein is associated with a reduction in lung, breast, and cervical cancer. In the vascular system, lutein is found in high-density lipoprotein (“HDL”) or “good” cholesterol and may prevent low-density lipoprotein (“LDL”) or “bad” cholesterol from oxidizing, which sets the cascade for heart disease.

Lycopene is an open-chain unsaturated carotenoid that imparts red color to tomatoes, guava, rosehip, watermelon, and pink grapefruit. Lycopene is a proven anti-oxidant that may lower the risk of certain diseases including cancer and heart disease. In the body, lycopene is deposited in the liver, lungs, prostate gland, colon, and skin. Its concentration in body tissues tends to be higher than all other carotenoids. Epidemiological studies have shown that high intake of lycopene-containing vegetables is inversely associated with the incidence of certain types of cancer. For example, habitual intake of tomato products has been found to decrease the risk of cancer of the digestive tract among Italians. In one six-year study by Harvard Medical School and Harvard School of Public Health, the diets of more than 47,000 men were studied. Of 46 fruits and vegetables evaluated, only the tomato products (which contain large quantities of lycopene) showed a measurable relationship to reduce prostate cancer risk. As consumption of tomato products increased, levels of lycopene in the blood increased, and the risk for prostate cancer decreased. Ongoing research suggests that lycopene can reduce the risk of macular degenerative disease, serum lipid oxidation, and cancers of the lung, bladder, cervix and skin. Studies are underway to investigate other potential benefits of lycopene including lycopene's potential in the fight against cancers of the digestive tract, breast, and prostate. W. Stahl & H. Sies, Lycopene: a biologically important carotenoid for humans? 336 Arch. Biochem. Biophys. 1-9 (1996); H. Gerster, The potential role of lycopene for human health, 16 J. Amer. Coll. Nutr. 109-126 (1997).

Alpha-lipoic acid (technically known as DL-alpha lipoic acid) is a powerful antioxidant being researched for unique properties that may provide both preventive and therapeutic benefits in numerous conditions and diseases including diabetes, heart disease, and possibly even HIV infection. Lipoic acid and its reduced form, DHLA, show the ability to directly quench a variety of reactive oxygen species, inhibit reactive oxygen generators, and spare and regenerate other antioxidants. Lipoic acid not only protects the nervous system, but is also involved in regenerating nerves. It is also being studied in the treatment of Parkinson's disease and Alzheimer's disease. Lipoic acid is best known for its ability to help regenerate damaged liver tissue when nothing else will. Lipoic acid is marketed in Germany for treating diabetic neuropathy. It also has an essential role in mitochondrial dehydrogenase reactions. Lipoic acid prevents and treats many age-related diseases, from heart disease and stroke to diabetes and cataracts.

Coenzyme Q10 is an essential electron and proton carrier that functions in the production of biochemical energy in aerobic organisms. Coenzyme Q10 is found in every cell in the body, thus its other name, ubiquinone (from the word ubiquitous and the coenzyme quinone). The structure of coenzyme Q10 consists of a quinone ring attached to an isoprene side chain. Because the body must have energy available to perform even the simplest operation, coenzyme Q10 is considered essential for the body's cells, tissues, and organs. Coenzyme Q10 also has antioxidant and membrane stabilizing properties that serve to prevent the cellular damage that results from normal metabolic processes. Even though the body has the ability to produce coenzyme Q10, deficiencies have been reported in a range of clinical conditions. Supplementation of the coenzyme helps guard against a possible deficiency. Aging is considered one reason for a deficiency, since the liver loses its ability to synthesize coenzyme Q10 as one gets older. Besides aging, poor eating habits, stress, and infection affect the body's ability to provide adequate amounts of coenzyme Q10. Known results of using coenzyme Q10 as an oral supplement are energy increase, improvement of heart function, prevention and cure of gum disease, a boost to the immune system, and possible life extension. AIDS is a primary target for research on coenzyme Q10 because of its immense benefits to the immune system. Further, coenzyme Q10 has also been reported to provide a salutary effect in the treatment of breast cancer.

Inositol is necessary for the formation of lecithin and functions closely with choline. Inositol is a fundamental ingredient of cell membranes and is necessary for proper functioning of nerves, brain, and muscles in the body. Inositol functions in conjunction with folacin, vitamin B-6 and vitamin B-12, choline, betaine, and methionine to prevent the accumulation of fats in the liver. Inositol is primarily used in the treatment of liver problems, depression, panic disorder, and diabetes. It is needed for health at the cellular level is concentrated in the lens of the human eye and in the heart. Men taking extra inositol reported that their hair loss decreased, although this has not been tested under clinical situations. Inositol plays an important part in the health of cell membranes especially the specialized cells in the brain, bone marrow, eyes, and intestines. The function of the cell membranes is to regulate the contents of the cells, which makes effective functioning possible. Inositol is said to promote healthy hair, hair growth, and helps in controlling estrogen levels and may assist in preventing breast lumps. It may also be of benefit in reducing blood cholesterol levels. If inositol intake is not sufficient, symptoms such as eczema, hair loss, constipation, abnormalities of the eyes, and elevated cholesterol levels may be experienced. No RDA has been established supplementation is usually 50 mg per day. No toxic effects known, but diarrhea has been noted with the intake of very high dosage of inositol. Inositol is best used with choline, B group vitamins, vitamin E, vitamin C, and linoleic acid.

Spirulina is a microalga that contains 60% all vegetable protein, essential vitamins and phytonutrients, betacarotene, the rare essential fatty acid gamma-linolenic acid (GLA), sulfolipids, glycolipids, and polysaccharides. Research has shown that taking spirulina extracts results in the tumor fighting ability of natural killer cells and gamma-interferon. In another study, spirulina was shown to be a potent inducer of gamma-interferon and a moderate inducer of interleukin-4 and interleukin-1 beta. Thus, spirulina strengthens the immune system and promotes immunity to intracellular pathogens and parasites. Other research suggested that spirulina has therapeutic effects on hyperlipidemia and obesity.

Other ingredients may also be added to the formulation. For example, fructose may be added as both an energy source and a sweetener. Fructose does not require insulin to enter certain cells of the body and therefore results in a smooth flow into the bloodstream and from there to the brain and other parts of the body. Low-calorie sweeteners, such as sucralose, may also be used according to the present invention. Flavors may be added to render the formulation more palatable. Synthetic flavors of almost any desired type are now available. Illustrative flavors that can be used according to the present invention include grape, strawberry, pineapple, and lemon flavors. In addition, certain organic acids may be added, such as citric acid, aspartic acid, tartaric acid, and malic acid. Aspartic acid is an amino acid and is used in building muscle. Citric acid is an acidulant, provides a pleasant tart flavor, and a component of effervescent powders. Malic acid is a flavoring agent, flavor enhancer, and acidulant. Tartaric acid is another acidulant and buffering agent.

The nutritional value of proteins in the human diet involves recognition of the quality as well as the quantity of the protein. Humans do not have the ability to synthesize all the amino acids required for normal good health. Those that are required to be supplied by the diet are called essential amino acids and include leucine, isoleucine, lysine, methionine, phenylalanine, threonine, tryptophan, and valine. In general, it is recommended that an adult should take in the daily diet 10 g of protein per kg of body weight. Children require about two to three times this amount. Of course, this assumes that the protein in the diet has an adequate amount of all essential and nonessential amino acids. Proteins found in eggs, beef, and milk are considered to have the best nutritional value.

Adequate protein nutrition requires the intake of sufficient protein to meet daily requirements. This protein must be of the necessary quality, i.e., supply the essential amino acids. Protein deficiency thus may be caused by a reduced intake or the use of low-quality protein. Obviously, the actual intake of protein may be influenced by factors such as high excretion in conditions of kidney damage or blood loss, or an increased requirement associated with thyrotoxicosis or high fever. Symptoms of deficiency include loss of weight, nutritional edema, and skin changes and are associated with such conditions as nephrosis, sprue, and colitis. Deficiency may result also in a reduced resistance to infection, since an adequate protein intake is necessary for the formation of phagocytes, leukocytes, and antibodies. Stress, such as brought on by accidental or surgical trauma, pregnancy, and lactation may also cause a deficiency of amino acids, and greater intakes of protein are required in these conditions.

Arginine is useful in enhancing the immune system, and it increases the size and activity of the thymus gland, which is responsible for manufacturing T lymphocytes, which are part of the immune system. Arginine is also important in liver health in that it assists in neutralizing ammonia. It is also involved in the skin and connective tissues, thus it is important in healing and repair of tissues, as well as the formation of collagen and building of new bone and tendons.

Cysteine is critical to the metabolism of a number of essential biochemicals, including coenzyme A, heparin, biotin, lipoic acid, and glutathione. Cysteine, which may be supplied as N-acetylcysteine, helps in strengthening the protective lining of the stomach and intestines. It is a constituent of the antioxidant, glutathione.

Glycine is required for building protein in the body and for synthesis of nucleic acids. Glycine has been found to be useful in aiding the absorption of calcium in the body. It is important for prostate health, and it is used by the nervous system as an inhibitory neurotransmitter, which is important for preventing epileptic seizures and for the treatment of bipolar disorder and hyperactivity.

Histidine is needed for growth and for the repair of tissue, as well as the maintenance of the myelin sheath, which acts as a protector for nerve cells. Histidine is also required for the manufacture of both red and white blood cells, and it helps to protect the body from damage caused by radiation and in removing heavy metals from the body. In the stomach, histidine is also helpful in producing gastric juices.

Isoleucine, together with the other two branched-chain amino acids, promotes muscle recovery after physical exercise. It is also needed for the formation of hemoglobin and for assisting with regulation of blood sugar levels and energy levels. It is also involved in blood clot formation.

Leucine helps with the regulation of blood-sugar levels, the growth and repair of muscle tissue, growth hormone production, wound healing, and energy regulation.

Lysine is required for growth and bone development in children, assists in calcium absorption, and assists in maintaining the correct nitrogen balance in the body and maintaining lean body mass. Further, lysine is needed to produce antibodies, hormones, enzymes, and collagen and to repair tissues.

Methionine assists in the breakdown of fats and thereby prevents the build-up of fat in the arteries. It also assists with proper functioning of the digestive system and for removing heavy metals from the body, since it can be converted to cysteine, a precursor to glutathione, which is of prime importance in detoxifying the liver. Methionine is also a great antioxidant, since the sulfur supplied in methionine inactivates free radicals. Methionine may also be used to treat depression, arthritis pain, and chronic liver disease. It is one of the three amino acids needed by the body to manufacture creatine, a compound essential for energy production and muscle building.

Phenylalanine is used for elevating mood, since it is closely involved with the nervous system. It also helps with memory and learning and has been used as an appetite suppressant.

Threonine is required to help maintain proper protein balance in the body, as well as assisting in formation of collagen and elastin in the skin. It is also involved in liver functioning (including fighting fatty liver), lipotropic functions-along with aspartic acid and methionine, and assisting in the immune system by helping the production of antibodies and promoting thymus growth and activity.

Tryptophan is required for the production of the vitamin, niacin. It is also used by the body to produce serotonin, a neurotransmitter that is important for normal nerve and brain function. Serotonin is important in sleep, stabilizing emotional moods, pain control, fighting inflammation, and maintaining intestinal peristalsis. It is also important in controlling hyperactivity in children, assisting in alleviating stress, helping with weight loss, and reducing appetite.

Valine is needed for and has a stimulating effect on muscle metabolism. It is also needed for repair and growth of tissue and maintaining the nitrogen balance in the body.

Additional objects and advantages of the present invention will be appreciated by one of ordinary skill in the art in light of the current description and examples of the preferred embodiments, and are expressly included within the scope of the present invention.

It is understood that the examples and embodiments described herein are for illustrative purposes only and that various modifications or changes in light thereof will be suggested to persons skilled in the art and are included within the spirit and purview of this application and are considered within the scope of the appended claims. All publications, patents, and patent applications cited herein are hereby incorporated by reference in their entirety for all purposes.

### EXAMPLE #1

Example of a Preferred Embodiment of the Nutritional Supplement of the Invention. In this particular embodiment of the nutritional supplement, a drink is prepared by adding the following ingredients directly to the volume of carrier, for example, distilled water, followed by performing the method of the invention.

| Herbal Extract | Amount |
|---|---|
| Rose flower water distillate | 0.25–20 g/L |
| (Damask Rose or Rose Mallow) | |
| Orange blossom flower distillate | 0.25–20 g/L |
| Jasmine, Chamomile, Tilia, Rose | 5–30 g/L |
| mallow, Chrysanthemum, Marigold, | (added to carrier as dried herbs) |
| Wolf Berry, Black Caraway, | |
| Cardamom, Mint, Sumac, Clove, Sage, | |
| Bergamot, Hyssop, Balm | |
| Lemon | 0–10 g/L |
| Lime | 0–10 g/L |
| Honey | 0–10 g/L |
| Sugar | 0–50 g/L |
| Salt | 0–50 g/L |

1. A process for preparing a nutrient supplement composition, comprising the steps of: (1) providing a volume of a carrier liquid; (2) providing a flower distillate component; (3) providing an herbal extract component; and (4) admixing the herbal extract, and the flower distillate with the liquid carrier, and optionally adding an additional ingredient selected from the group consisting of a nutrient, a flavoring agent, a preservative, a coloring agent, a second carrier agent, and combinations thereof.

2. The process of claim 1, wherein the step of providing an herbal extract further comprises the steps of: providing a suitable amount of an herb; providing a volume of a carrier liquid; adding the herb to the carrier liquid and heating the carrier liquid to a temperature of from about 40° C. to about 200° C. for a duration of from about 10 seconds to about one hour; steeping the herbal component in the carrier liquid for a duration of from about 1 minute to about 48 hours, wherein heat is no longer applied to the carrier liquid; and filtering the herb pulp from the carrier liquid.

3. The process of claim 2, wherein the amount of herb comprises from about 0.5 grams to about 60 grams per liter of carrier liquid.

4. A method for preparing a nutritional supplement composition, comprising the steps of: (1) providing a volume of a carrier liquid; (2) providing a flower distillate component; (3) providing a suitable amount of an herb; (4) placing the herbal component in the carrier liquid and heating to a temperature of from about 40° C. to about 200° C. for a duration of from about 10 seconds to about one hour; (5) steeping the herbal component in the carrier liquid for a duration of from about 1 minute to about 48 hours, wherein heat is no longer applied to the carrier liquid; and (6) filtering the herbal component pulp from the carrier liquid, and wherein the flower distillate is added to the carrier liquid either prior to boiling or after filtering.

5. The process of claim 4, wherein the amount of herb comprises from about 0.5 grams to about 60 grams per liter of carrier liquid.

6. The process of claim 1, wherein the carrier comprises at least one of water, alcohol, oil, or a combination thereof.

7. The process of claim 2, wherein the carrier comprises at least one of water, alcohol, oil, or a combination thereof.

8. The process of claim 1, wherein the flower distillate comprises at least one of rose flower water, orange blossom water or a combination thereof.

9. The process of claim 2, wherein the herb comprises at least one member selected from the group consisting of chamomile, orange blossom, tilia, marigold, wolf berry, rose mallow, chrysanthemum, black caraway, cardamom, mint, sumac, clove, lemon, lime, sage, bergamot, hyssop, jasmine, and balm.

10. The process of claim 1, wherein the nutrient comprises at least one member selected from the group consisting of mineral, calcium, magnesium, chromium, copper, iodine, iron, manganese molybdenum, selenium, zinc, boron, sodium, potassium, silicon, carotenoids, beta-carotene, lutein, zeaxanthin, lycopene, choline, para-aminobenzoic acid, alpha-lipoic acid, flavonoids, coenzyme Q10, an oil, an essential oil, a lipid, a phospholipid, a salt, linoleic acid, linolenic acid, inositol, methylsulfonyl methane, spirulina, a vitamin, vitamin A, vitamin C, vitamin D, vitamin E, vitamin K, vitamin B-12, thiamine, riboflavin, niacin, pantothenic acid, pyridoxine, folic acid, biotin, derivatives thereof, an amino acid, arginine, histidine, lysine, isoleucine, leucine, methionine, phenylalanine, threonine, tryptophan, tyrosine, valine, aspartate, glutamate, serine, proline, asparagine, glutamine, cysteine, glycine, and alanine.

11. The process of claim 1, wherein the flavoring agent comprises at least one member selected from the group consisting of anethole, anise oil, benzaldehyde, blackberry, blueberry, caraway, caraway oil, cardamom oil, cardamom seed, cherry juice, cherry syrup, cinnamon, cinnamon oil, an alcohol, cinnamon water, citric acid, citric acid syrup, clove oil, cocoa, coriander oil, dextrose, eriodictyon, ethyl acetate, ethyl vanillin, fennel oil, ginger, glucose, glycerin, glycyrrhiza, grape, honey, lavender oil, lemon oil, lime, mannitol, methyl salicylate, myristica oil, orange oil orange peel, orange syrup, peppermint, peppermint oil, peppermint water, phenylethyl alcohol, pineapple, raspberry juice, raspberry syrup, rosemary oil, rose oil, rose water, sarsaparilla syrup, sorbitol, spearmint, spearmint oil, strawberry, sucrose, fructose, fruit juice, thyme oil, tolu balsam, vanilla, vanillin, and wild cherry syrup.

12. The process of claim 2, wherein the herb comprises dried or fresh herb.

13. A nutritional supplement composition, comprising a carrier liquid, a flower distillate component, an antioxidant containing herbal extract component, and at least one member selected from the group consisting of a nutrient, a flavoring agent, a preservative, a coloring agent, a second carrier agent, and combinations thereof.

14. (canceled)

15. The nutritional supplement of claim 13, wherein the carrier liquid comprises at least one of water, oil, alcohol or a combination thereof.

16. The nutritional supplement of claim 13, wherein the flower distillate comprises at least one of rose flower water, orange blossom water or a combination of both.

17. The nutritional supplement of claim 13, wherein the antioxidant containing herbal extract comprises an extract of at least one member selected from the group consisting of chamomile, orange blossom, tilia, marigold, wolf berry, rose mallow, chrysanthemum, black caraway, cardamom, mint, sumac, clove, lemon, lime, sage, bergamot, hyssop, jasmine, and balm.

18. (canceled)

19. The nutritional supplement of claim 13, wherein the nutrient comprises at least one member selected from the group consisting of a mineral, calcium, magnesium, chromium, copper, iodine, iron, manganese molybdenum, selenium, zinc, boron, sodium, potassium, silicon, an antioxidant, carotenoids, beta-carotene, lutein, zeaxanthin, lycopene, choline, para-aminobenzoic acid, alpha-lipoic acid, flavonoids, coenzyme Q10, an oil, an essential oil, a lipid, a salt, a phospholipid, linoleic acid, linolenic acid, inositol, methylsulfonyl methane, spirulina, a vitamin, vitamin A, vitamin C, vitamin D, vitamin E, vitamin K, vitamin B-12, thiamine, riboflavin, niacin, pantothenic acid, pyridoxine, folic acid, biotin, derivatives thereof an amino acid, arginine, histidine, lysine, isoleucine, leucine, methionine, phenylalanine, threonine, tryptophan, tyrosine, valine, aspartate, glutamate, serine, proline, asparagine, glutamine, cysteine, glycine, and alanine.

20. The nutritional supplement of claim 13, wherein the flavoring agent comprises a member selected from the group consisting of anethole, anise oil, benzaldehyde, blackberry, blueberry, caraway, caraway oil, cardamom oil, cardamom seed, cherry juice, cherry syrup, cinnamon, cinnamon oil, an alcohol, cinnamon water, citric acid, citric acid syrup, clove oil, cocoa, coriander oil, dextrose, eriodictyon, ethyl acetate, ethyl vanillin, fennel oil, ginger, glucose, glycerin, glycyrrhiza, grape, honey, lavender oil, lemon oil, lime, mannitol, methyl salicylate, myristica oil orange oil, orange peel, orange syrup, peppermint, peppermint oil, peppermint water, phenylethyl alcohol, pineapple, raspberry juice, raspberry syrup, rosemary oil, rose oil, rose water, sarsaparilla syrup, sorbitol, spearmint, spearmint oil, strawberry, sucrose, fructose, fruit juice, thyme oil, tolu balsam, vanilla, vanillin, and wild cherry syrup.

21. The nutritional supplement of claim 13, wherein the supplement is in the form of a carbonated liquid.
